(19) United States
(12) Patent Application Publication
(10) Pub. No.: US 2011/0172515 A1
(43) Pub. Date: Jul. 14, 2011

(54) DYNAMIC CORRECTION OF HIGH FREQUENCY ADJUSTMENT DURING PARALLEL TRANSMISSION

(76) Inventors: Hans-Peter Fautz, Forchheim (DE); Franz Schmitt, Erlangen (DE)

(21) Appl. No.: 12/986,828

(22) Filed: Jan. 7, 2011

(30) Foreign Application Priority Data
Jan. 13, 2010 (DE) 10 2010 004 514.4

(51) Int. Cl. A61B 5/055

(52) U.S. Cl. 600/410

(57) ABSTRACT

The present embodiments relate to a system and a method for operating an imaging system, where a plurality of subvolumes of an examination volume of an examination object to be examined with the system is examined. The examination volume is assembled from the plurality of subvolumes, where to examine the subvolumes, at least one HF pulse is transmitted in each case. The at least one HF pulse is optimized for the subvolume that is to be examined therewith respect to specifications and basic conditions applicable for the subvolume.

This application claims the benefit of DE 10 2010 004 514.4, filed Jan. 13, 2010.

### BACKGROUND

The present embodiments relate to a system and a method for operating an imaging system.

U.S. Pat. No. 7,218,113 B2 and DE 10 2004 002 009 B4 describe a method for operating a magnetic resonance system, in which a B1 field distribution is measured in at least one subregion of an examination volume of a high-frequency antenna of the magnetic resonance system, and on the basis of the determined B1 field distribution, the HF pulses emitted by the high-frequency antenna are optimized for homogenization in a specific volume. An effective volume within the examination volume is determined beforehand for each applied RF pulse, and on the basis of the determined B1 field distribution, the relevant RF pulse is individually adjusted such that the B1 field is homogenized within the effective volume of the RF pulse. The high-frequency antenna may include a plurality of antenna elements, the antenna elements being selectively controlled with a particular phase and a particular amplitude for each HF pulse, such that the homogeneity of the B1 field generated overall by the HF pulses is maximized in the effective volume of the HF pulse or an optimization volume located therein (the intersection of effective volume and ROI).

U.S. Pat. No. 7,242,193 B2 and DE 10 2005 017 310 B4 describe a method for generating a high-frequency magnetic field, used for spin excitation in an examination volume, in the interior of a cylindrical body coil of a magnetic resonance apparatus. The body coil includes a plurality of resonator segments distributed around the circumference and a control apparatus for separate activation of the individual resonator segments, electromagnetically decoupled from one another. The resonator segments are activated such that the magnetic field is generated only in at least one first subvolume forming the examination volume, and at least one second subvolume that is not to be excited, is essentially free of the magnetic field.

DE 102009020661.2 describes how, within an imaging sequence, the volume to be mapped is recorded and subdivided into subvolumes (e.g., during 2D imaging in a plurality of layers or during 3D imaging in a plurality of “slabs”). With respect to the effective volume, different attributes may be automatically optimized by the control apparatus, since the effective volume is known. The amplitude of the high-frequency pulse to be transmitted and the frequency emitted by the NCO may be simultaneously optimized. A further subsequence that follows on directly from a first subsequence is a chemical saturation (e.g., a fat saturation).

In conventional MR imaging using one transmission channel, the transmission profile is constant right down to a global phase and cannot be temporally altered. Only the excitation profile (e.g., the generated transverse magnetization) may be spatially modulated by the simultaneous impact of HF and gradient pulses on the spin system. The spatially selective modulations, however, result in long pulse times and an inefficient use of the HF pulses: the average tilt angle per irradiated output is reduced.

By simultaneously and independently operating a plurality of transmission coils, the resulting B1 field may, with an adjustment of the phases and amplitudes of each individual transmitter, be varied spatially and temporally in phase and amplitude. The phases and amplitudes are calculated using suitable pulse design algorithms, such that the magnetization generated after the pulse approximates a predefined target magnetization as closely as possible. The spatial distribution of the target magnetization is thus a mandatory condition on the pulse design. Other mandatory conditions (specifications) may be, for example, the minimization of the absorbed overall output (global SAR), the spatial distribution of the absorbed output (local SAR), and the required maximum output. The BO field distribution and spectral composition in the examination object, motion of magnetization and different tissue characteristics such as relaxation time likewise condition the pulse design problem and may be taken into account as additional mandatory conditions.

The quality of the resulting pulse (measured against the target specifications) depends on the number of mandatory conditions and the number of (independent) degrees of freedom. For example, “better” pulses may be generated with more HF transmission coils or longer HF pulses that are temporally modulated on the transmission channels independently of one another. A higher number of channels is, however, associated with additional costs and a more complex system of safety monitoring. Longer pulses that simultaneously modulate the HF envelope and gradients may be more susceptible to artifacts and to lose efficiency (as explained above).

A small target volume may be used to ease the mandatory conditions on the pulse design. In other words, within a small subvolume of the overall volume covered by the transmission coils, target specifications with respect to the generated magnetization and/or the specific absorption rate (SAR) may be satisfied more precisely and more easily. This has until now been utilized for local imaging, in which the volume to be examined was small overall, such as in prostate imaging (see e.g., B. van den Bergen et al., “SAR and Power Implications of Different RF Shimming Strategies in the Pelvis for 7T MRI,” Journal of Magnetic Resonance Imaging, Vol. 30, No. 1, 2009: pp. 194-202).

With the help of mandatory conditions and regulations, results may be generated in numeric optimization methods that weight different target specifications very flexibly. For example, the accuracy of the achievable target magnetization may be increased very simply and almost continuously at the expense of the specific absorption rate. In other words, there is a lot of room for maneuver for adjusting compromises to a particular application or question. However, the spatial distribution of target magnetizations in large volumes places heavy demands on the pulse design. Depending on the resolution, many thousands of voxels, in each case, impose mandatory conditions. The restriction of the target volume for pulse optimization has, until now, only been known for small examination volumes.

### SUMMARY AND DESCRIPTION

The present embodiments may obviate one or more of the drawbacks or limitations in the related art. For example, the imaging in an imaging system may be optimized.

### BRIEF DESCRIPTION OF THE DRAWINGS

FIG. 1 schematically shows one embodiment of an MRT system.

### DETAILED DESCRIPTION OF THE DRAWINGS

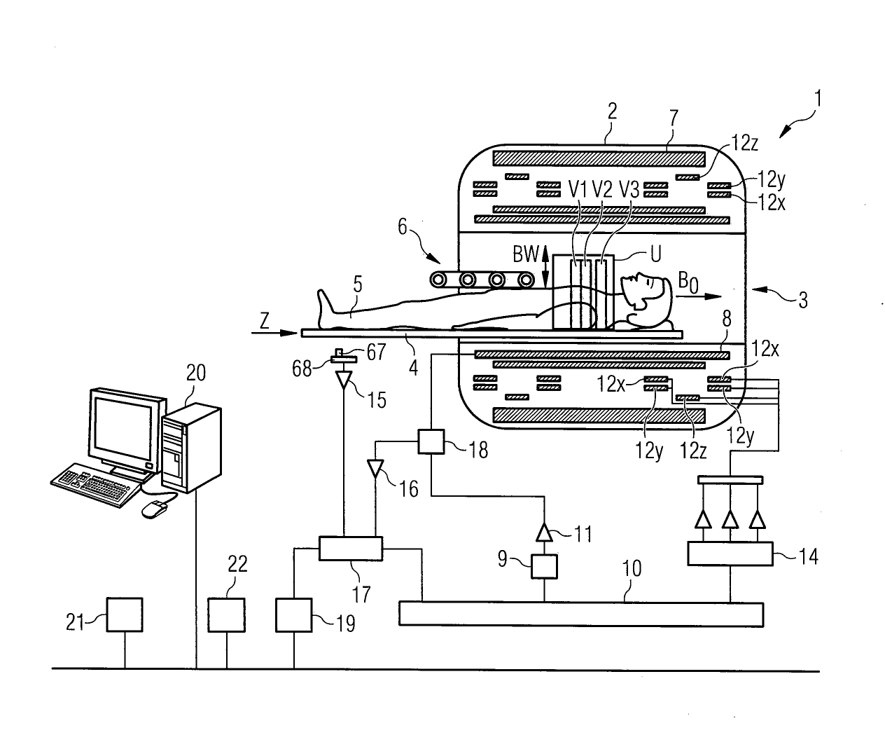

FIG. 1 shows an imaging magnetic resonance device MRT 1 with a whole-body coil 2. The whole-body coil 2 includes a tube-shaped space 3, into which a patient couch 4 holding a body (e.g., of a patient 5) may be introduced in the direction of the arrow z (with or without local coil arrangement 6), in order to generate recordings of the patient 5. A local coil arrangement 6, with which recordings are facilitated in a local region (i.e., a field of view), is imposed on the patient here. Signals from the local coil arrangement 6 may be evaluated (e.g. converted into images and stored or displayed) by an evaluation apparatus (e.g., including elements 67, 66, 15, 17) of the MRT 1, which may be connected by coaxial cable or by radio, for example, to the local coil arrangement 6.

In order to examine the body 5 using the magnetic resonance device MRT 1 using magnetic resonance imaging, different magnetic fields, aligned with one another as precisely as possible with regard to temporal and spatial characteristics of the different magnetic fields, are radiated onto the body 5.

A strong magnet such as, for example, a cryomagnet 7 in a measuring booth with the tunnel-shaped opening 3, generates a static strong main magnetic field B₀. The main magnetic field may be 0.2 tesla to 3 tesla or more, for example. The body 5 to be examined is positioned on the patient couch 4 and introduced into an approximately homogeneous region of the main magnetic field 7 in the field of view U.

An excitation of the nuclear spin of atomic nuclei of the body 5 is effected using magnetic high-frequency excitation pulses that are radiated via a high-frequency antenna (and if necessary, a local coil), represented in a simplified manner in FIG. 1 as a body coil 8. High-frequency-excitation pulses are generated by a pulse generation unit 9 that is controlled by a pulse sequence control unit 10. After amplification by a high-frequency amplifier 11, the high-frequency-excitation pulses are routed to the high-frequency antenna 8. The high-frequency system shown in FIG. 1 is shown schematically. More than one pulse generation unit 9, more than one high-frequency amplifier 11 and a plurality of high-frequency antennas 8 may be used in the magnetic resonance device 1.

The magnetic resonance device 1 also includes gradient coils 12 x, 12 y, 12 z, with which, during a measurement, magnetic gradient fields are radiated for selective layer excitation and for position encoding of the measured signal. The gradient coils 12 x, 12 y, 12 z are controlled by a gradient coil control unit 14 that, as in the case of the pulse generation unit 9, is connected to the pulse sequence control unit 10.

The signals transmitted by the excited nuclear spin are received by the body coil 8 and/or at least one local coil arrangement 6, amplified by associated high-frequency pre-amplifiers 16 and further processed and digitized by a receiver unit 17. The measured data recorded is digitized and stored as complex numerical values in a k-space matrix. An associated MR image may be reconstructed from the k-space matrix populated with values using a multidimensional Fourier transformation. In the case of a coil that may be operated both in the transmit and in the receive mode (e.g., the body coil 8), the correct forwarding of signals is regulated by an upstream duplexer 18.

An image processing unit 19 generates, from the measured data, an image that is displayed to a user via an operating console 20 and/or is saved in a storage unit 21. A central computing unit 22 controls the individual system components.

In MR tomography, images with a high signal/noise ratio (SNR) may be recorded using (local) coils. The coils are antenna systems that are placed in the immediate vicinity on (anterior) or under (posterior) the patient. In the case of MR measurement, the excited nuclei induce a voltage in the individual antennas of the local coil. The induced voltage is amplified with a low-noise pre-amplifier (e.g., LNA, Preamp) and passed to the receiving electronics by cable, for example. To improve the signal/noise ratio even in high-resolution images, high-field systems are used (e.g., 1.5 T and greater). Since more individual antennas may be connected to an MR receiving system than the number of receivers present, a switching matrix (e.g., RCCS) is built in between the receiving antennas and receiver. This routes the currently active receiving channels (e.g., mostly the receiving channels lying directly in the field of view of the magnet) to the receivers. As a result, more coil elements than the number of receivers present may be connected, since with whole-body coverage, only those coils that are located in the field of view (FoV) or in the homogeneity volume of the magnet need be read out.

The “local coil arrangement” 6 may be an antenna system that may, for example, include one antenna element (coil element) or a plurality of antenna elements (coil elements) in the form of an array coil. These individual antenna elements may be designed as loop antennas (loops), or butterfly or saddle coils. A local coil arrangement includes coil elements, the pre-amplifier, further electronics (e.g., sheath wave traps) and wiring, the housing and for example, a cable with plug, by which the local coil arrangement is connected to the MRT system. A receiver 68 on the system side filters and digitizes the signal received (e.g., by radio) from the local coil 6 and passes the data to digital signal processing, which from the data obtained by a measurement, may derive an image or a spectrum and make the image or the spectrum available to the user (e.g., for subsequent diagnosis by the user or for storage).

According to the present embodiments, an examination volume U may be subdivided into a plurality of subvolumes V1, V2, V3. For each subvolume of the plurality, all HF pulses that are used for recording (or imaging with respect to) the subvolume are established (i.e., defined) and optimized in each case. For HF pulse optimization, a target magnetization is predefined as a specification during the establishment of optimized HF pulses only for the respective subvolume. For signal generation within each subvolume, the associated HF pulses (e.g., the HF pulses established as optimized for the subvolume) are used. Signals from the plurality of subvolumes are generated and recorded temporally nested or in sequence for recording the overall examination volume. In this case, the HF pulse applied is dynamically adapted to the subvolume recorded in each case.

The recording of a large examination volume in a plurality of subvolumes is known in magnetic resonance tomography. A volume is, for example, recorded in a plurality of parallel shims (e.g., a plurality of sectors) in multilayer recordings, with only signals in one shim being generated and read in each case in each recording act. Consecutive recording steps in the same shim may be temporally nested with the neighboring shims or sequentially recorded.

Methods are known that assemble a large volume out of a plurality of parallel three-dimensional blocks (e.g., slabs) that are recorded in a similar way to the shims in multilayer recordings. The present embodiments are directly applicable to such methods: the HF pulses are optimized separately for each shim or 3D layer and are applied shim-specifically during the data recording.

One embodiment includes selecting the layer management using criteria of HF pulse optimization. The variation in the B1 field distributions may be much less along the z axis (e.g., the axis of the main magnetic field) than in a transverse plane lying perpendicular to the z axis. Layer management along the z axis may thus be helpful in optimizing the homogeneity in different regions of the transverse plan on a time-staggered basis.

Another embodiment includes subdividing the overall volume using pulse optimization criteria. Thus, for example, in breast imaging, it may be helpful to record the left and right breast in two separate volume datasets rather than in one. If the corresponding layer management is chosen, these may be recorded temporally nested. Any loss of signal-to-noise ratio because of the reduced recording steps per subvolume may in some cases be equalized by simultaneously extending the repetition time.

Subdividing the overall volume U into the plurality of subvolumes V1, V2, V3 includes the option of choosing subvolumes that are irregular in size and shape. Spatially selective pulses in conjunction with parallel transmission offer the possibility of being able to select any subvolumes.

The overall optimization process may be represented schematically as follows: (1) segmentation of the overall volume into suitable subvolumes; (2) HF pulse optimization for each subvolume; and (3) signal generation and image recording, with a pulse optimized for the current subvolume being selected for each timepoint.

It is not only with respect to the subvolume currently being recorded that the HF pulses may be established on an optimized basis and selected correspondingly during the recording. It is known for basic conditions such as, for example, physiological procedures such as respiration or heartbeat to alter the BO and B1 field. HF pulses for basic conditions such as different motion statuses during respiration or heartbeat may be optimized. The present embodiments may also provide for HF pulses for different timepoints of a motion cycle (e.g., respiration, heartbeat) to be optimized with respect to BO, B1 and a motion. FIG. 1 shows, by way of example of a motion, a respiratory motion in the form of the arrow BW in the breast region of the patient 5. The status of the motion cycle may be logged using, for example, a respiratory belt, EKG and/or navigators. The pulses optimized for the respective timepoint are transmitted during the data recording.

The whole optimization process for establishing pulses may be represented as follows: (1) measuring the time-dependent BO and/or B1 fields; (2) pulse optimization for different field distributions; (3) association of the various HF pulses with timepoints within the motion cycle; and (4) signal generation and image recording, with an optimized pulse being selected for each timepoint.

One embodiment includes optimizing HF pulses for different timepoints during the image recording and dynamically adapting the signal generation to the currently pertaining basic conditions.

With conventional single-channel systems, this is possible only to a limited extent (i.e., with the help of complex pulses that simultaneously vary the HF envelope and the gradient amplitude).

With multichannel transmission systems, the B1 field distributions of HF pulses may be selectively altered and temporally adapted. The complex pulses described, which modulate magnetization profiles, will also find wider diffusion because of these systems than was previously the case.

In the present embodiments, the establishment of the HF pulses for the subvolume currently to be examined in each case may bring a significant gain in homogeneity (or more precisely, targeted excitation profile) plus a significant reduction in the SAR. As a result, local imaging is performed at each timepoint. The mandatory conditions (e.g., specifications) for pulse optimization such as, for example, target magnetizations or SAR values—are limited to the subvolume currently to be examined and thus significantly reduced. For example, specifications with respect to target magnetizations within the subvolume currently to be examined may be achieved much more precisely or with a significantly reduced SAR. These advantages may be further expanded by selecting the subvolumes using pulse optimization criteria.

A reduced incidence of artifacts in the pulses or in the generated signals may be expected as a result of the temporal adjustment of the HF pulses to physiologically conditioned changes in the measured spin ensemble.

The following are examples of considerations included in a pulse optimization method: HF shimming; spatially selective pulses (simultaneous impact of HF and gradients); selection of the subvolumes; prior knowledge (e.g. transmission profiles of the coils in z and x, y); test optimizations for different segmentations (e.g. HF shimming); and analysis of the transmission profiles: Separate the areas with largest local variances.

A possible advantage of the present embodiments is to use homogeneity, more precise target magnetization and reduced SAR well for large examination volumes.

Another possible advantage is to measure the temporal change of mandatory conditions such as the BO or B1 field distribution (e.g., on the basis of respiratory or heart motion) to adapt the pulse calculation to the temporal changes and to correct the application of the pulses such that at every timepoint, an optimum pulse is used for the current subvolume and magnetization status.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for operating an imaging system, the method comprising:
examining a plurality of subvolumes of an examination volume of an examination object to be examined with the imaging system; and
assembling the examination volume from the subvolumes,
wherein examining the plurality of subvolumes comprises:
establishing a high frequency (HF) pulse for each subvolume of the plurality, taking into account specifications and conditions for the subvolume; and
transmitting the HF pulse for each subvolume of the plurality.

2. The method as claimed in claim 1, further comprising:
establishing the HF pulses for a plurality of timepoints during the examination of the examination volume,
wherein establishing the HF pulses comprises optimizing the HF pulse for the subvolume to be examined and the timepoint at which the HF pulse is transmitted with respect to specifications, conditions or specifications and conditions for the subvolume and the timepoint.

3. The method as claimed in claim 1, wherein a period in which an examination of a subvolume of the plurality takes place at least partially overlaps temporally with a period in which an examination of another subvolume of the plurality takes place.

4. The method as claimed in claim 1, wherein for each subvolume of the plurality, a plurality of HF pulses that is used for the examination of the subvolume is optimized for the subvolume.

5. The method as claimed in claim 1, wherein when establishing the HF pulse for a subvolume of the plurality, a target magnetization predefined only for the subvolume is taken into account.

6. The method as claimed in claim 1, wherein the examination volume is subdivided into the plurality of subvolumes along an axis of a B0 magnetic field or B1 magnetic field of the imaging system.

7. The method as claimed in claim 1, wherein the examination volume is subdivided into the plurality of subvolumes, one subvolume of the plurality being spatially separated from another subvolume of the plurality.

8. The method as claimed in claim 1, further comprising optimizing HF pulses for different timepoints of a motion cycle of the examination object during the examination of the examination volume, the optimization taking into account conditions present at the different timepoints.

9. The method as claimed in claim 1, wherein for the examination of a subvolume of the plurality at a timepoint, the HF pulse that is optimized for the subvolume and the timepoint is used.

10. The method as claimed in claim 1, wherein establishing the HF pulse comprises optimizing an amplitude, phase, the progression of amplitude, the progression of phase, or combinations thereof of the HF pulse.

11. The method as claimed in claim 1, wherein the specifications taken into account include magnetization to be generated in the subvolume by the HF pulse, overall magnetization to be generated in the subvolume, a maximum specific absorption rate (SAR) in the subvolume, or combinations thereof.

12. The method as claimed in claim 2, wherein the conditions taken into account include at least conditions of the examination object obtained on the basis of a model of the examination object, of measured values during a measurement of the examination object with or without preparation pulses, or combinations thereof.

13. The method as claimed in claim 2, wherein the conditions taken into account include at least one gradient field to be transmitted at the timepoint, one B0 field present at the timepoint, the HF pulse, or combinations thereof.

14. The method as claimed in claim 1, wherein the imaging system is a magnetic resonance tomography device.

15. The method as claimed in claim 1, wherein the examination volume is subdivided into the plurality of subvolumes using pulse optimization criteria.

16. The method as claimed in claim 1, wherein the examination volume is subdivided into the plurality of subvolumes, such that the subvolumes are operable to be shimmed.

17. The method as claimed in claim 1, wherein the examination volume is subdivided into the plurality of subvolumes along an axis approximately perpendicular to the direction along which the examination object is introduced into the imaging system.

18. The method as claimed in claim 16, wherein the plurality of subvolumes are shimmed such that a specific absorption rate (SAR) in each subvolume of the plurality is minimized.

19. The method as claimed in claim 18, wherein different shimmings of the subvolumes are calculated for the subvolumes, and
wherein the shimming with the best SAR in the subvolume is selected for each subvolume of the plurality.

20. An imaging system comprising:
a control apparatus configured such that a plurality of subvolumes of an examination volume of an examination object to be examined with the imaging system are examined;
a pulse optimization apparatus configured to establish high frequency (HF) pulses, each of the HF pulses being optimized for one respective subvolume of the plurality with respect to specifications and conditions for the one subvolume; and
an HF pulse transmission apparatus configured to transmit the HF pulses established by the pulse optimization apparatus,
wherein at least one HF pulse is transmitted to examine each subvolume of the plurality.

21. The system as claimed in claim 20, wherein the HF pulses are optimized for a plurality of timepoints during the examination of the examination volume, and
wherein to examine a subvolume of the plurality, at least one HF pulse that is optimized for the subvolume to be examined and the timepoint at which the HF pulse is transmitted with respect to specifications and conditions for the subvolume and the timepoint, is transmitted.

22. The system as claimed in claim 20, wherein a period in which a subvolume of the plurality is examined at least partially overlaps temporally with a period in which an examination of another subvolume takes place.

23. The system as claimed in claim 20, wherein for each subvolume of the plurality of subvolumes, a plurality of HF pulses that are used for the examination of the subvolume are all optimized for the subvolume.

24. The system as claimed in claim 20, wherein a target magnetization predefined only for a subvolume of the plurality is taken into account when determining at least one HF pulse for the subvolume.

25. The system as claimed in claim 20, wherein the examination volume is subdivided into the plurality of subvolumes along an axis of a B1 magnetic field of the imaging system.

26. The system as claimed in claim 20, wherein the examination volume is subdivided into the plurality of subvolumes, such that the plurality of subvolumes are separated from each other in spatially separate regions of the examination object.

27. The system as claimed in claim 20, wherein HF pulses for different timepoints of a motion cycle of the examination object are optimized during the examination of the examination volume, taking into account conditions present at the different timepoints, the different timepoints being during respiratory or heart motions of the examination object.

28. The system as claimed in claim 20, wherein one of the HF pulses is used to examine each subvolume of the plurality at a timepoint that is optimized for the subvolume and the timepoint.

29. The system as claimed in claim 20, an amplitude, phase, the progression of amplitude, the progression of phase, or combinations thereof of an HF pulse is optimized when the HF pulse is determined.

30. The system as claimed in claim 20, wherein specifications for a subvolume of the plurality to be examined with one of the HF pulses are taken account of to determine the one HF pulse, and
wherein the specifications include magnetization to be generated in the subvolume by the at least one HF pulse, overall magnetization to be generated in the subvolume, a maximum specific absorption rate (SAR) in the subvolume, or combinations thereof.

31. The system as claimed in claim 20, wherein the conditions to be taken into account when determining the HF pulse for a subvolume of the plurality, a timepoint, or the subvolume and the timepoint include conditions of the examination object obtained on the basis of a model of the examination object, measured values during a measurement of the examination object with or without preparation pulses, or the model of the examination object and the measured values.

32. The system as claimed in claim 20, wherein the conditions to be taken into account when determining an HF pulse for a subvolume of the plurality, a timepoint, or the subvolume and the timepoint include at least one gradient field to be transmitted at the timepoint, one B0 field present at the timepoint, the HF pulse, or combinations thereof.

33. The system as claimed in claim 20, wherein the imaging system is a magnetic resonance tomography device.

34. The system as claimed in claim 20, wherein the establishment of the HF pulses taking into account the specifications and conditions for the one subvolume is the optimization of the one HF pulse taking into account the specifications and conditions for the one subvolume.

35. The system as claimed in claim 20, wherein the examination volume is subdivided using pulse optimization criteria.

36. The system as claimed in claim 20, wherein the examination volume is subdivided such that the plurality of subvolumes is shimmed.

37. The system as claimed in claim 20, wherein the examination volume is subdivided along an axis perpendicular to an axis of the imaging system or approximately perpendicular to the direction along which an examination object is introduced into the imaging system.

38. The system as claimed in claim 20, wherein the plurality of subvolumes are shimmed such that the SAR in the plurality of subvolumes is minimized.

39. The system as claimed in claim 20, wherein different shimmings of the plurality of subvolumes are calculated for the plurality of subvolumes, and
wherein a shimming is selected for each subvolume of the plurality.

40. An imaging system comprising:
a control apparatus configured such that a plurality of subvolumes of an examination volume of an examination object to be examined with the imaging system are examined;
a pulse optimization apparatus configured to establish high frequency (HF) pulses, the HF pulses being optimized for one subvolume of the plurality with respect to specifications and conditions for the one subvolume; and
an HF pulse transmission apparatus configured to transmit the HF pulses established by the pulse optimization apparatus.
